(19) United States
(12) Patent Application Publication
(10) Pub. No.: US 2024/0268999 A1
(43) Pub. Date: Aug. 15, 2024

(54) SYSTEMS AND METHODS FOR CREATING CHANNELS IN AN EYE USING LASER PULSES FOR THE TREATMENT OF GLAUCOMA

(71) Applicant: Christopher Horvath, San Juan Capistrano, CA (US)

(72) Inventor: Christopher Horvath, San Juan Capistrano, CA (US)

(21) Appl. No.: 18/442,850

(22) Filed: Feb. 15, 2024

Related U.S. Application Data
(60) Provisional application No. 63/485,010, filed on Feb. 15, 2023.

(51) Int. Cl. A61F 9/007; A61F 9/008

(52) U.S. Cl. CPC A61F 9/00781; A61F 9/0084; A61F 2009/00865

(57) ABSTRACT

Methods and devices for treating target tissue layers of an anterior chamber angle of an eye, using novel photo disruptive laser pulses delivered to multiple target tissue layers of the eye and thereby creating openings and channels in said tissue layers that create additional outflow channels for Aqueous humor out of the eye and thereby reducing the intraocular pressure of the eye and therefore treating Glaucoma.

### CROSS-REFERENCES TO RELATED APPLICATIONS

This application claims the benefit of U.S. Patent Application No. 63/485,010, filed on Feb. 15, 2023, the entirety of which is incorporated herein by reference.

### BACKGROUND AND BRIEF SUMMARY OF THE INVENTIONS

Lasers have been used for several decades in the treatment of glaucoma. The 2 most common laser treatments for primary open angle glaucoma (POAG) are ALT (Argon Laser Trabeculoplasty) and SLT (Selective Laser Trabeculoplasty). See for example U.S. Pat. Nos. 3,884,236; 8,066,696; 5,549,596; 6,319,274. They work by applying laser pulses into the Trabecular Meshwork (located in the anterior angle of the eye). These laser pulses are focused to around 50 micrometer diameter for ALT and around 400 micrometer for SLT. Those laser spots are targeted to lay over the Trabecular meshwork and increase outflow through the treated meshwork area into the Schlemm's canal by modulating the tissue properties. In both procedures at least 90 degrees of the anterior angle of the eye is treated with typically 180 degrees and 50 to 100 laser pulses (each pulse is applied to a new target zone-treatment area). The working mechanism for ALT is blanching of the Trabecular meshwork that increases the outflow by stretching the Trabecular Meshwork between the blanched (laser treated areas). The ALT laser with a typical setting of 600 mW and 0.1 s pulse duration (at 514 nm or 532 nm) causes a thermal tissue interaction. In SLT treatment the laser causes cavitation bubbles in the target tissue due to its shorter pulse duration of about 3 nanoseconds and higher peak power (created by pulse energies of around 0.3 mJ to 1.6 mJ).

Both procedures have a good success rate by increasing aqueous humor outflow that creates a substantial drop in intraocular pressure of around 20%. Both procedures can be performed in minutes with a simple slit lamp procedure in the office (no operating room required). In both procedures, the eye does not need to be opened (non-invasive procedure, no incisions needed), therefore the treatment risks and complication rates are minimal. The problem of these procedures as published in many studies is that it does not work effectively in all patients and in the successful cases the effect wears off over the course of a few years (1-3 years) and the IOP rises overtime. The procedure can be repeated once with ALT and 2-3 times with SLT, but after those repeats the tissue damage in the Trabecular meshwork that is created through those multiple procedures ultimately prevents any further IOP lowering effect.

A less frequently used laser procedure called ELT (Excimer Laser Trabeculostomy) uses an Excimer laser pulse (wavelength in the UV range) to actually drill holes into the Trabecular Meshwork. See for example U.S patent applications: 20080082078; 20040082939. Because complete openings are created to Schlemm's canal (unlike ALT and SLT), the IOP lowering effect is similar or better than ALT/SLT and in the same time only a few open holes need to be drilled with ELT versus 50-100 treatment zones in a typical ALT/SLT procedure. Some studies further suggest that the ELT effect is longer lasting then ALT/SLT due to some observed long-term patency of those holes. Furthermore, ELT might be repeated more often since a smaller area of the Trabecular Meshwork is treated each time. The downside of ELT is the fact that UV wavelength light does not penetrate the cornea and aqueous humor, therefore the laser can only be applied to the Trabecular Meshwork in an sterile operating room, where the eye is opened and a fiber probe is inserted into the anterior chamber all the way up to the Trabecular Meshwork.

In recent years the effectiveness of having one or multiple holes in the Trabecular Meshwork (connecting to Schlemm's canal) has also been demonstrated with several implants, placed through the Trabecular Meshwork that creates a connection of the anterior chamber to Schlemm's canal, bypassing the meshwork. Another surgical method to remove, cut or incise part or all of the Trabecular meshwork is called Goniotomy or Trabeculotomy often done by inserting a mechanical device into the eye. See for example U.S patent applications: 20120071809, 20070276316. Those are however also invasive (sterile operating room required) procedures using an implant or a surgical tool inside the eye.

Another approach to drain aqueous humor out of the anterior chamber has been successfully demonstrated by implanting a drainage tube through the scleral spur region and into the suprachoroidal space. See for example U.S patent application: 20110098629. This is however also an invasive (sterile operating room required) procedures using an implant.

Most recently, there have been animal tissue studies and initial human trials done by ViaLase applying ultrashort photodisruptive laser pulses to the trabecular meshwork with good success. See for example: Vialase in Opthtalmology Times, Sep. 21, 2021.

All here described current laser methods of treating glaucoma in an eye are effective by either modulating the tissue layers of the Trabecular Meshwork (ALT and SLT) or by creating one or multiple holes in the Trabecular meshwork (ELT or femto-glaucoma laser) to increase the outflow capacity of aqueous humor out of the anterior chamber of the eye.

The following here presented inventions go beyond these approaches and use laser pulses for a variety of novel glaucoma treatments.

### BRIEF DESCRIPTION OF THE DRAWINGS

Various features of illustrative embodiments of the inventions are described below with reference to the drawings. The illustrated embodiments are intended to illustrate, but not to limit, the inventions. The drawing contain the following figures:

FIG. 1 is a laser system block diagram

FIG. 2 is a portion of the laser system block diagram

FIG. 3 is a portion of the laser system block diagram

FIG. 4 is a portion of the laser system block diagram

FIG. 5 is a portion of the laser system block diagram

FIG. 6 shows the basic anatomy of an eye

FIG. 7 shows the basic anatomy of an eye

FIG. 8 shows an eye with a sheet and a lens on top of the cornea

FIG. 9 shows an eye with a lens on top of the cornea

FIG. 10 shows an eye with a basic patient interface on top

FIG. 11 shows an eye with a basic patient interface on top

FIG. 12 shows an eye with a lens on top of the cornea

FIG. 13 shows an eye with a lens on top of the cornea

FIG. 14 shows an eye with a lens on top of the cornea

FIG. 15 shows an eye with a lens on top of the cornea

FIG. 16 shows an eye with a lens on top of the cornea and a limbus diagnostic area

FIG. 17 shows an eye with a lens on top of the cornea and a camera

FIG. 18 shows an eye with a lens on top of the cornea and a camera

FIG. 18b illustrates a 3D scanning system

FIG. 19 shows an eye with a lens on top of the cornea and a camera

FIG. 20 shows an eye with a lens on top of the cornea and a camera

FIG. 21 shows an eye with a lens on top of the cornea and a camera

FIG. 22 shows an eye with a lens on top of the cornea and a camera

FIG. 23 shows an eye with a lens on top of the cornea and a camera

FIG. 24 shows an eye with a lens on the cornea, a camera and a laser beam

FIG. 25 shows an eye with a lens on the cornea, a camera and a laser beam

FIG. 26 shows an eye with a lens on the cornea, a camera and a laser beam and various delivery system optics

FIG. 27 shows the anatomy of the anterior angle area of an eye with a collapsed Schlemm's canal

FIG. 28 shows the anatomy of the anterior angle area of an eye with Schlemm's canal in takt

FIG. 29 Shows an opening created through the Trabecular Meshwork

FIG. 30 a,b,c shows various laser scanning patterns

FIG. 31 Shows a laser scanning direction back to front

FIG. 32 Shows a circular scanning pattern

FIG. 33 shows a laser treatment opening the Trabecular Meshwork and part of a collector channel

FIG. 34 shows a laser treatment opening the Trabecular Meshwork and opening a channel into the scleral tissue

FIG. 35 shows a laser treatment opening the Trabecular Meshwork and opening a channel into the scleral tissue

FIG. 36 shows a laser treatment opening through the scleral spur and opening a channel into the scleral tissue

FIG. 37 shows a laser treatment opening through the scleral spur and opening a channel into transition zone between the sclera and the choroid

FIG. 38 illustrates all previous described target tissue areas in one drawing

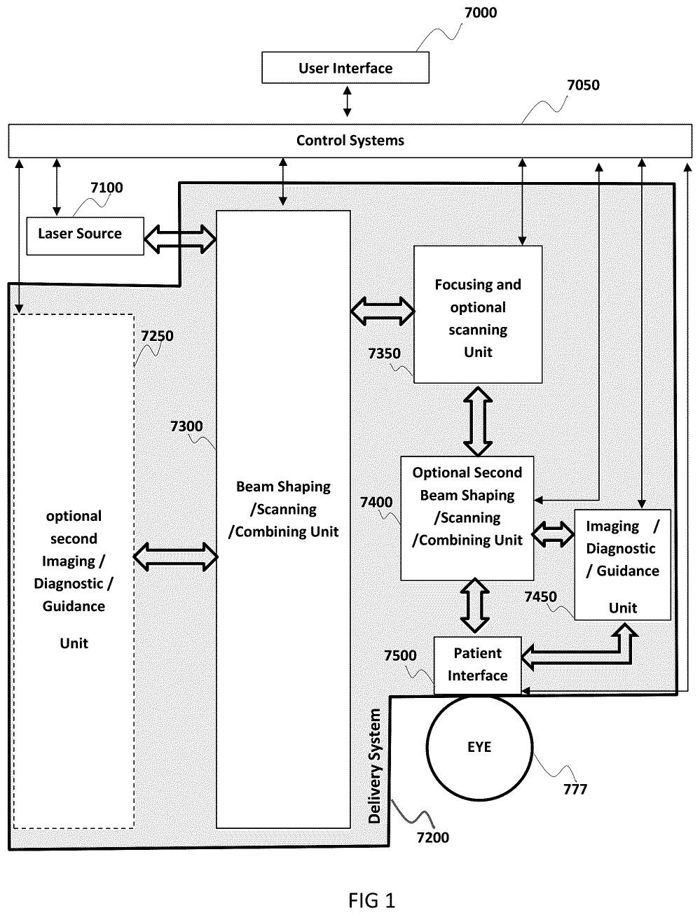

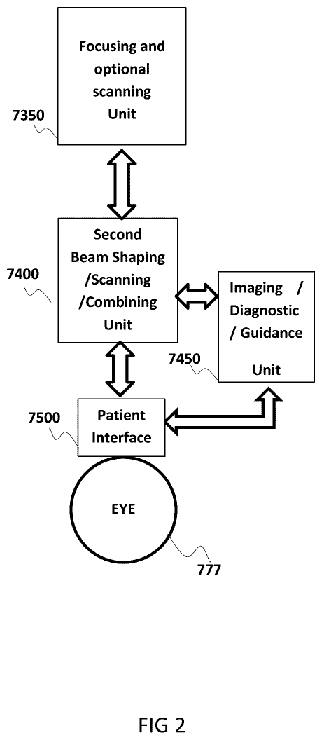

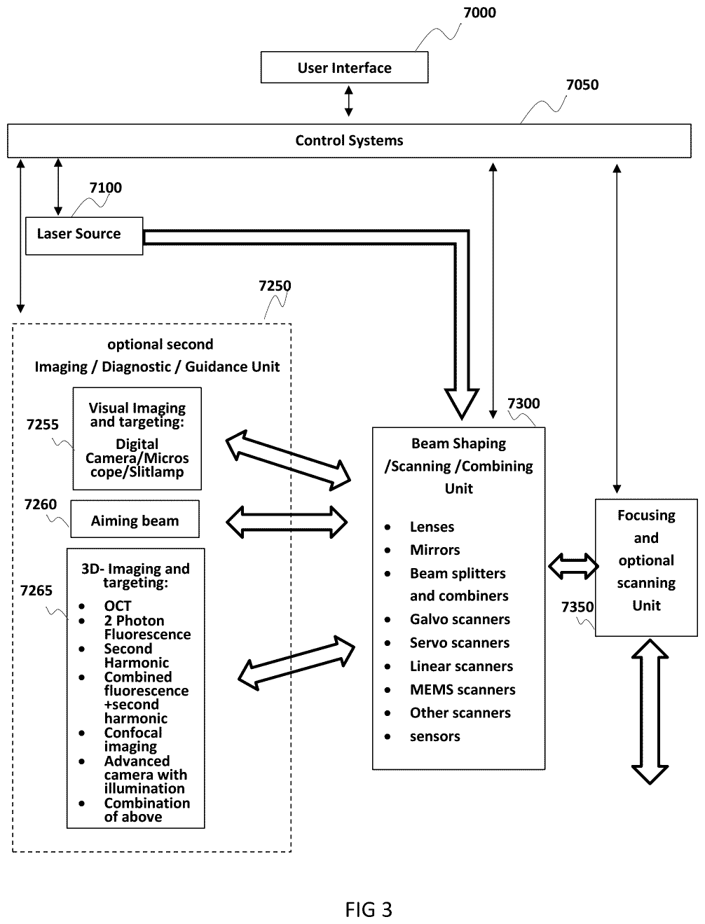

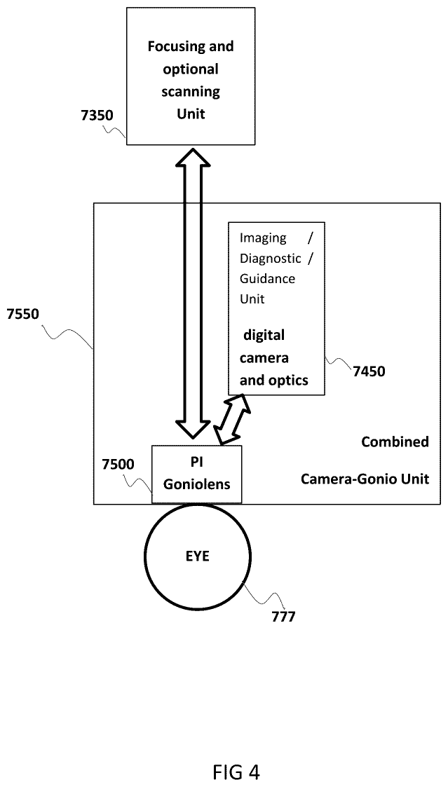

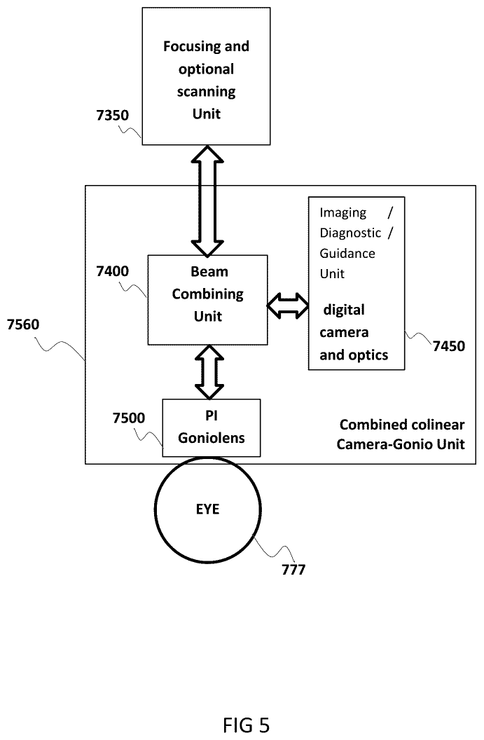

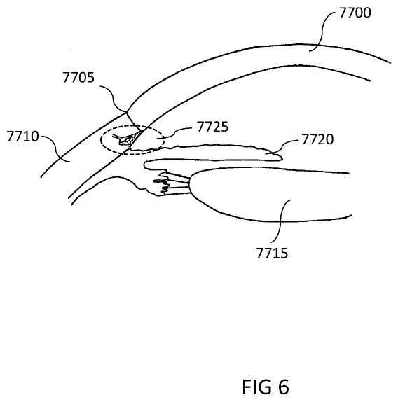

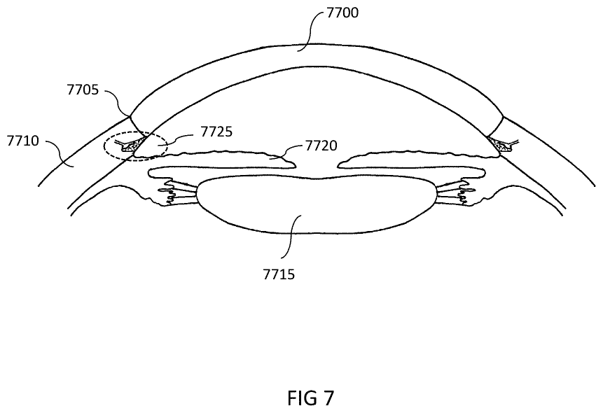

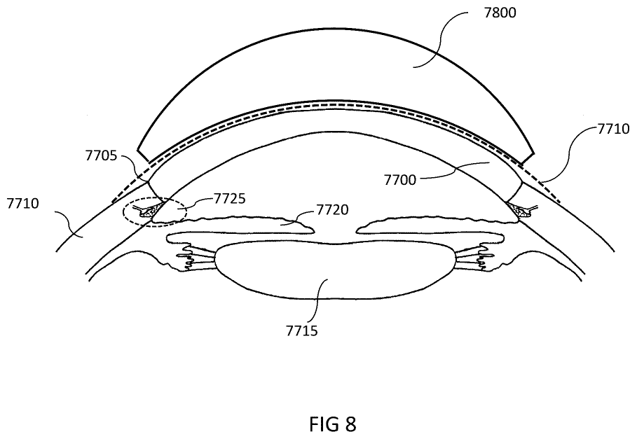

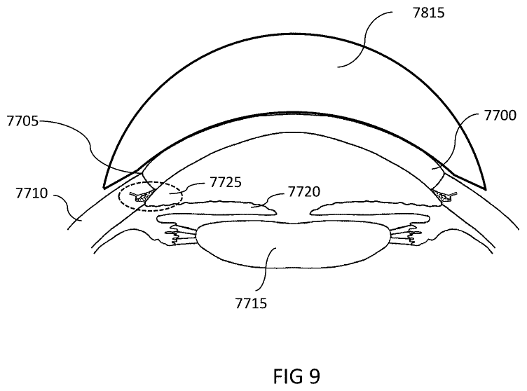

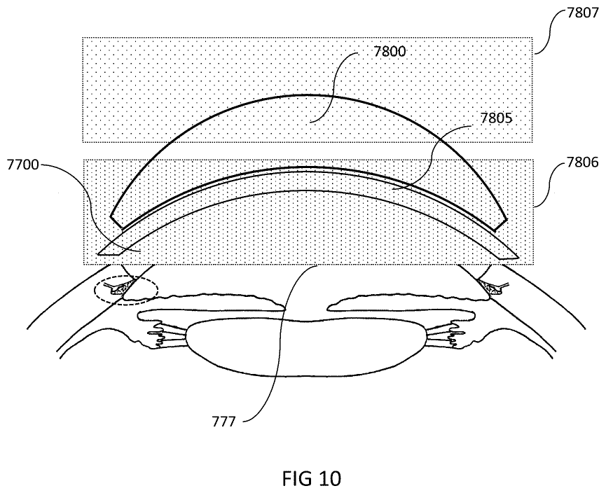

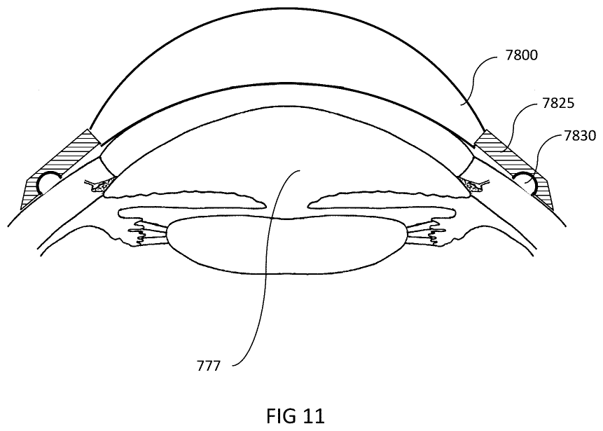

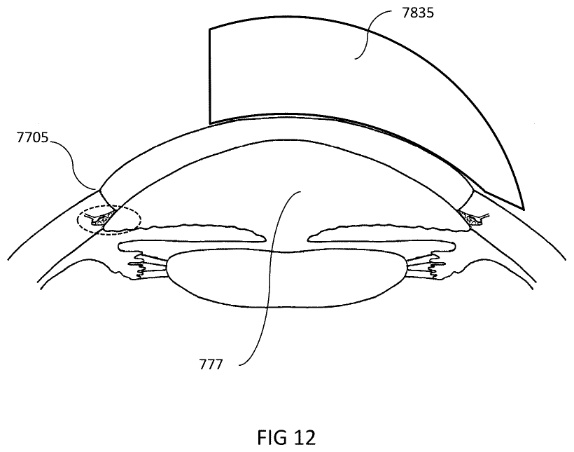

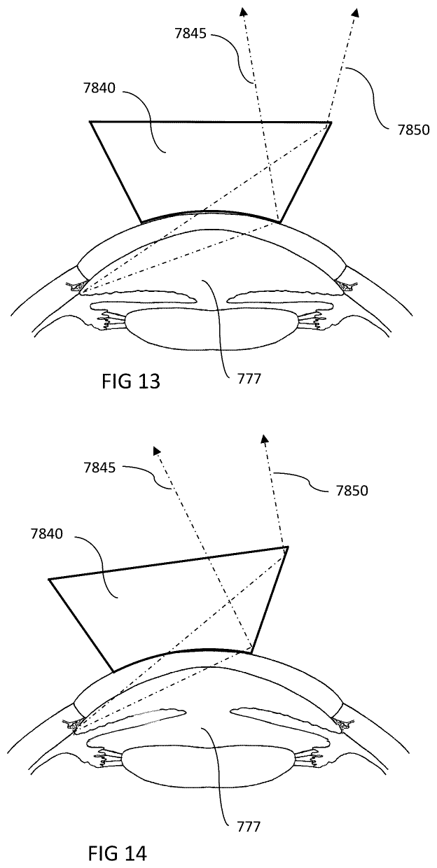

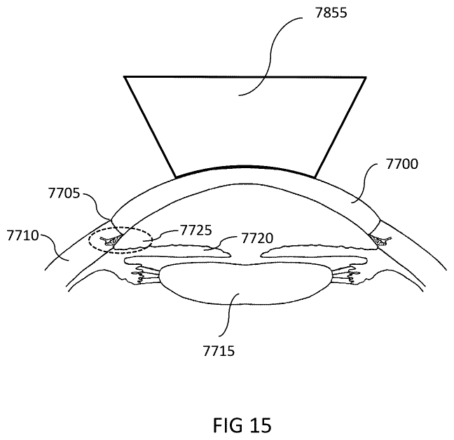

### DETAILED DESCRIPTION OF THE INVENTIONS AND CLAIMS

The following chapters describe 5 novel Methods for laser eye surgery as well as several novel laser systems to perform such procedures.

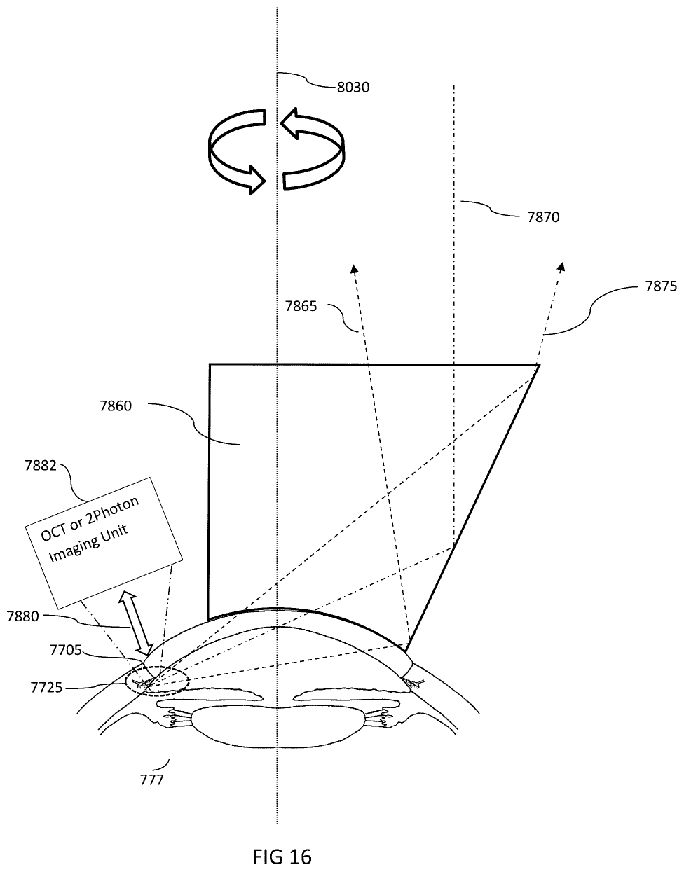

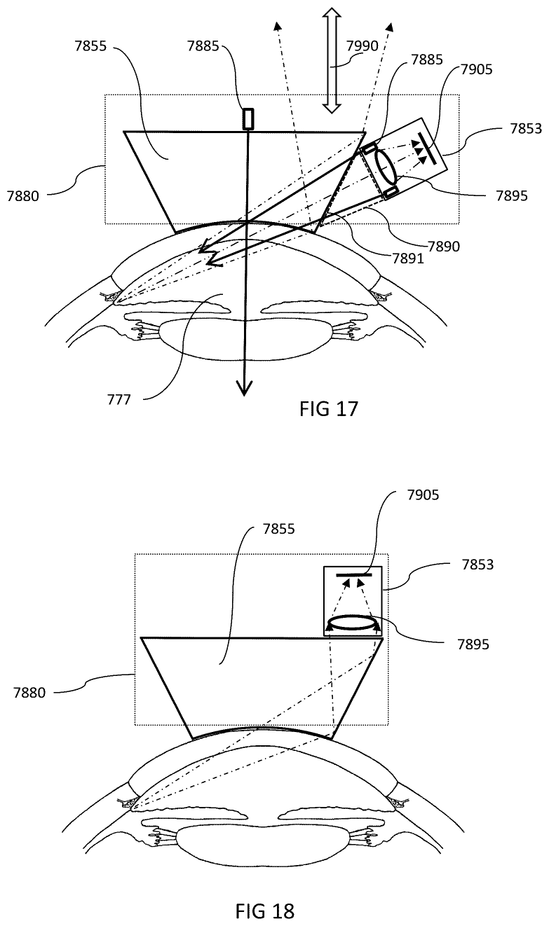

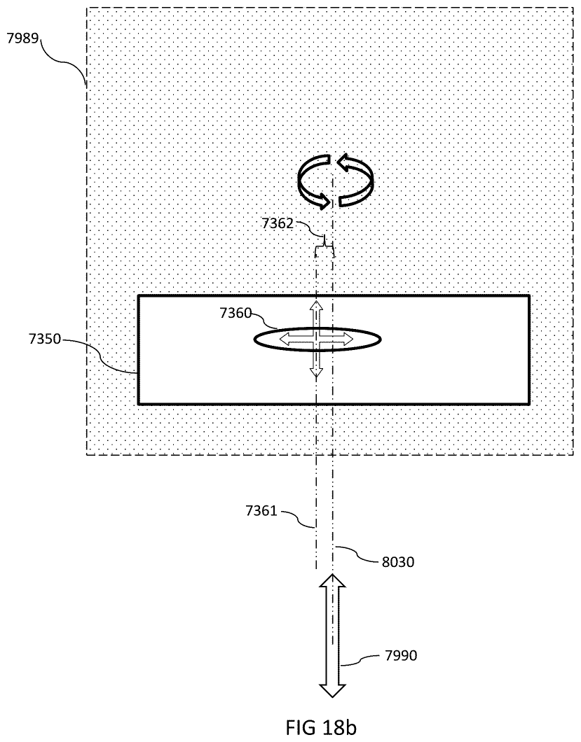

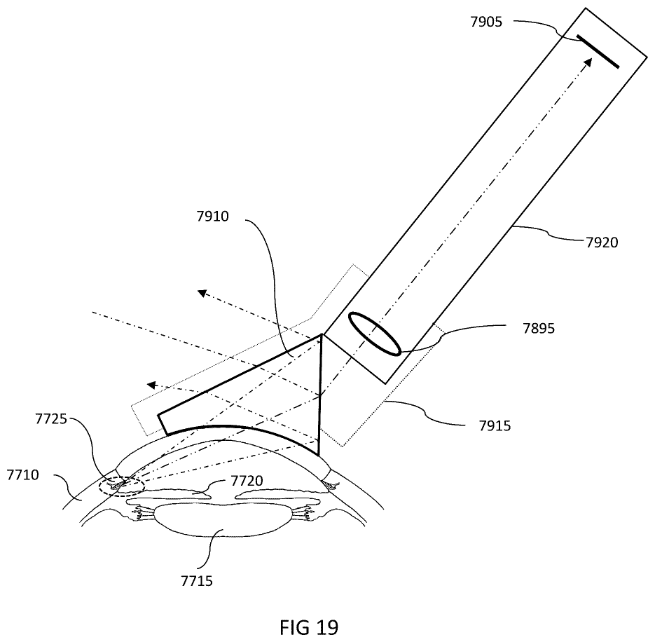

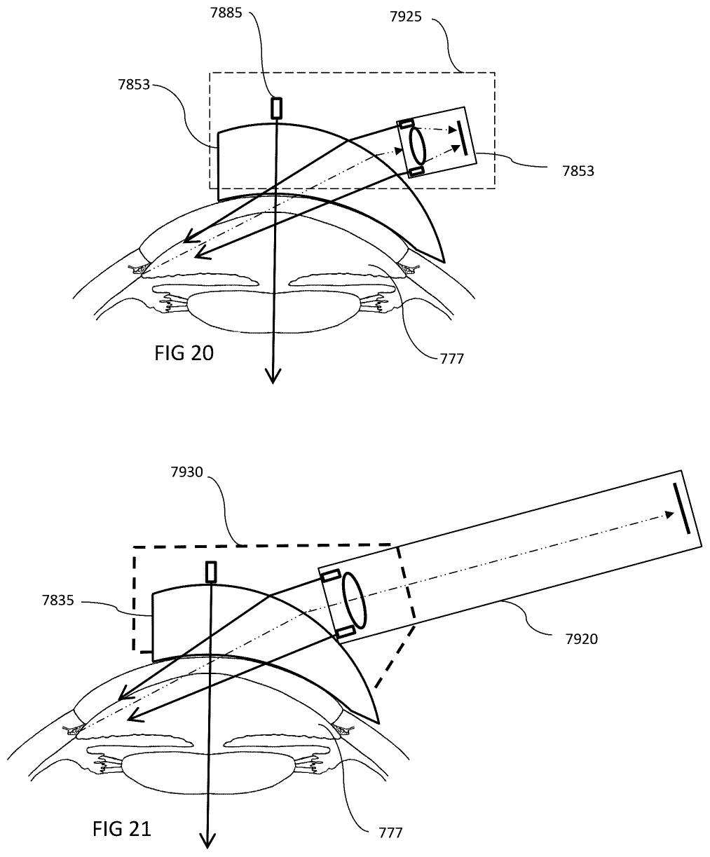

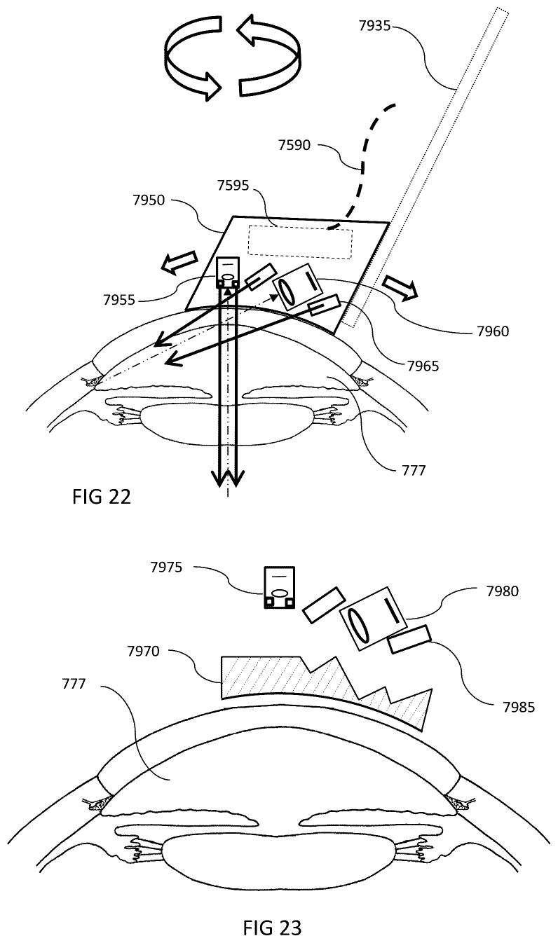

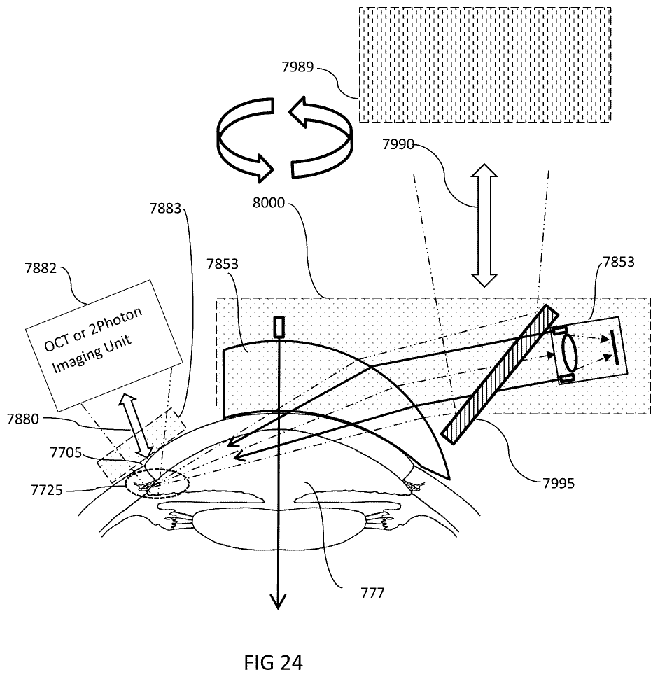

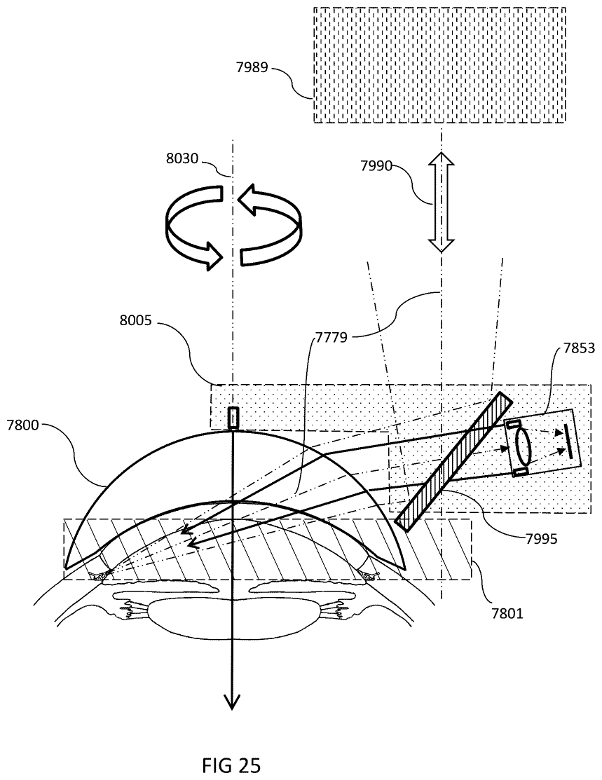

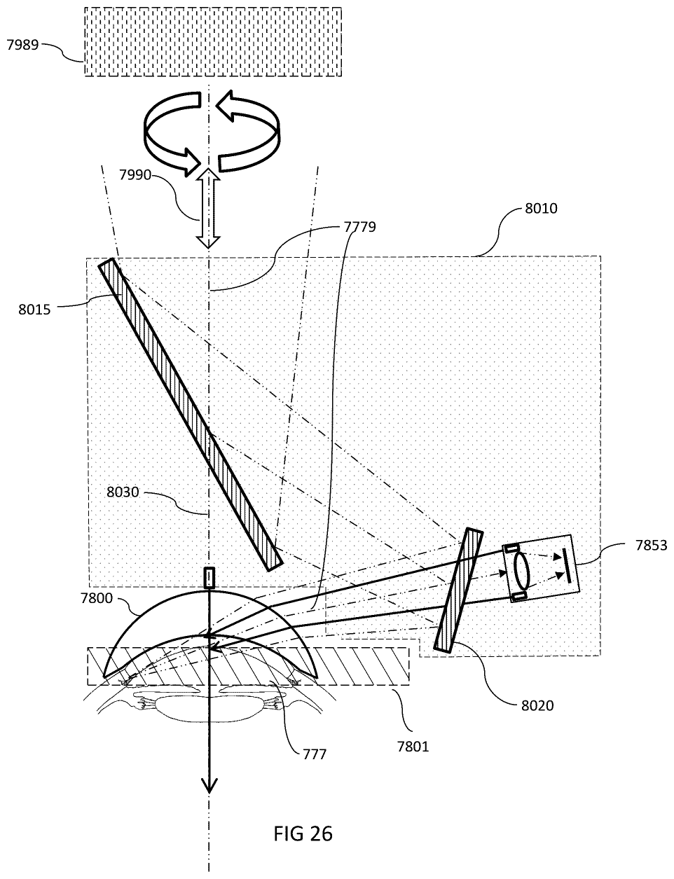

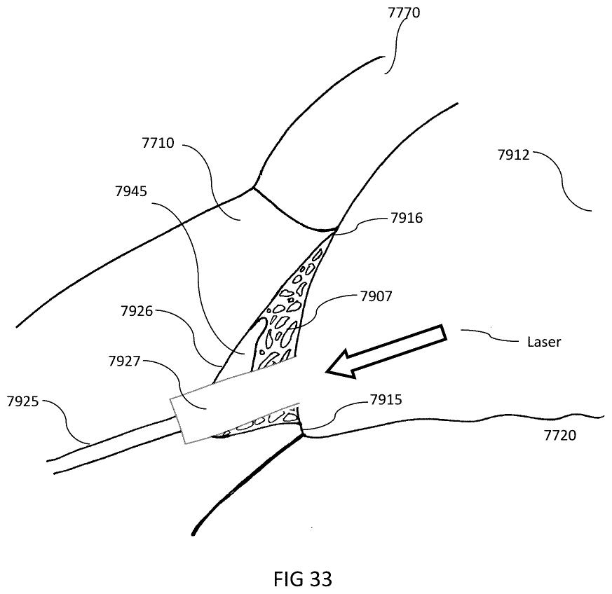

In all further described methods and systems, the laser treatment beam enters the eye through the cornea e.g. see FIG. 16 or FIG. 25 and then is being focused on the target tissue layers by propagating within the eye towards an anterior angle region of the eye in its longitudinal direction. This laser propagation is such that e.g. FIG. 33 the Trabecular Meshwork 7907 is the proximal end of the tissue target layers and e.g. Schlemm's Canal 7945 or a collector channel 7925 here in FIG. 33 become the distal end of the tissue target layers and thereby also of the laser treatment beam propagation. The laser treatment beam focus propagates thereby “ab interno” from the inside of the eye (proximal end) towards the outside of the eye (distal end). The immediate area around the laser focus is where the fluence of the laser pulse exceeds the photodisruptive threshold of the tissue it is focused on and therefore the focus area is were the tissue is locally disrupted and by scanning said laser focus over multiple laser pulses around the target tissue area, channels, holes and other 3D shapes can be cut into the target tissue layers, therefore allowing e.g. outflow of Aqueous Humor into different opened up tissue structures as described below.

### 1) Optimizing Outflow by Identifying and Targeting Collector Channels in the Eye to Treat Glaucoma:

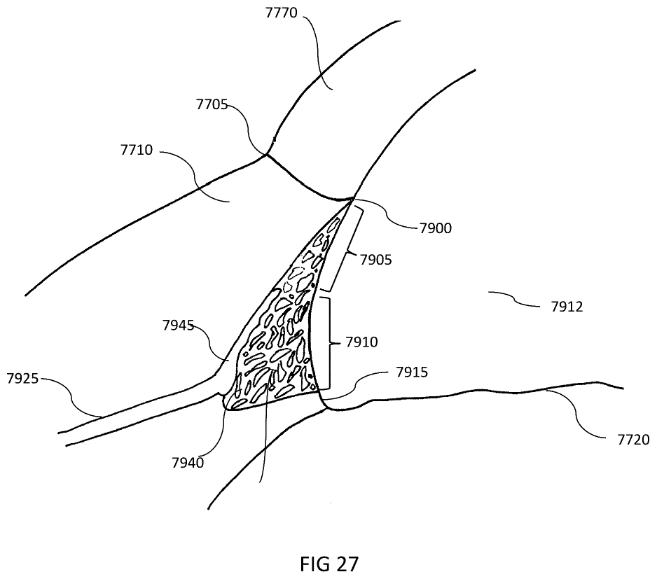

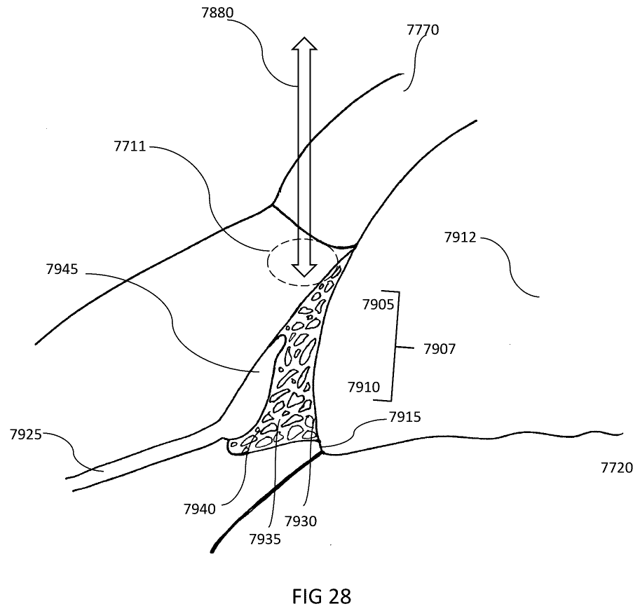

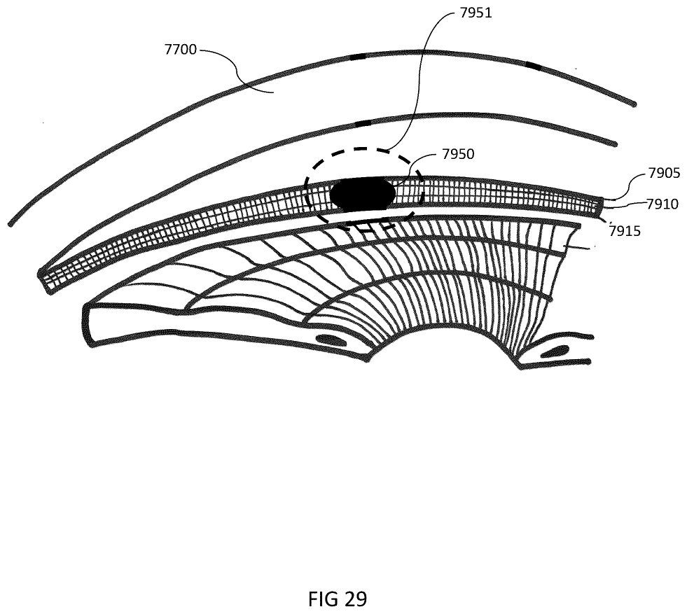

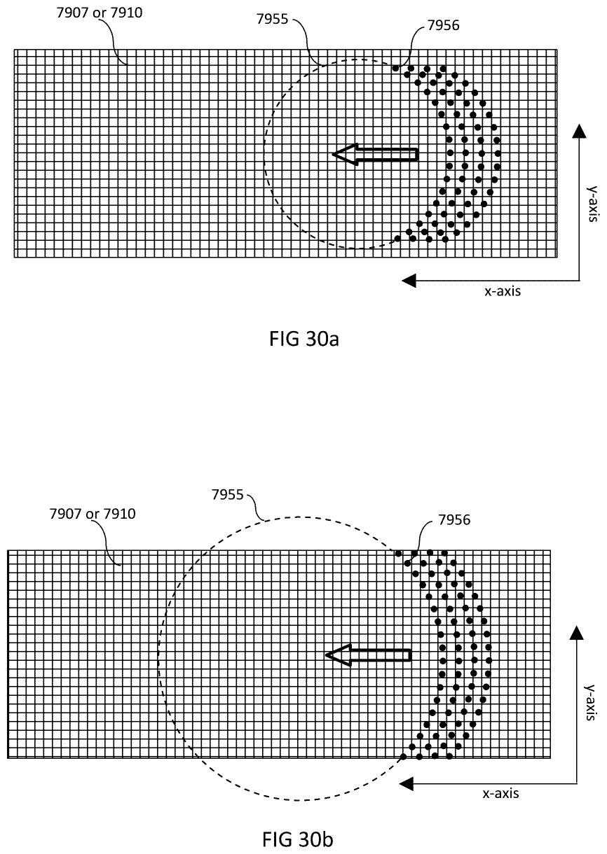

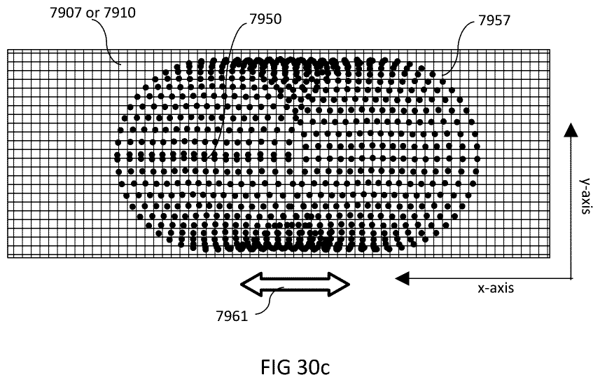

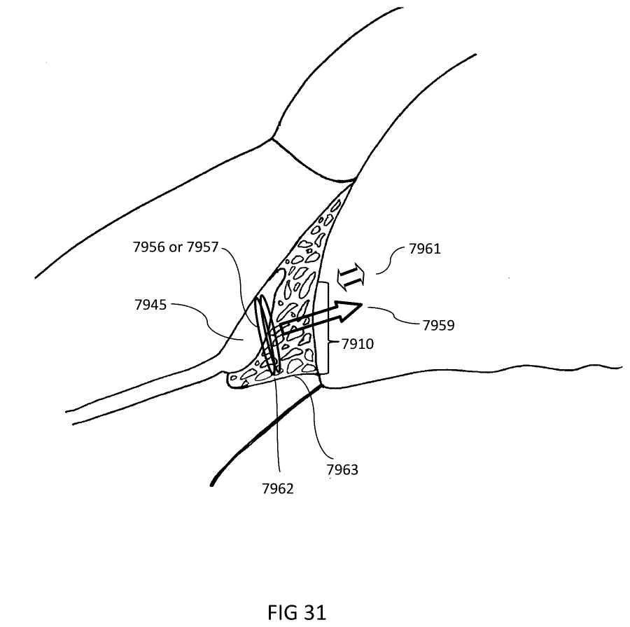

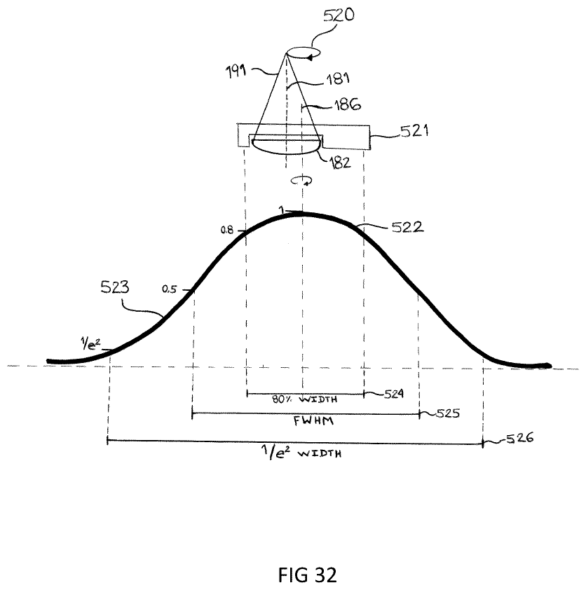

As shown in FIG. 28, in a healthy or early stage glaucoma eye the aqueous humor that fills the anterior chamber 7912 flows through the trabecular meshwork 7907 into Schlemms canal 7945 and out through the 20-30 Collector channels 7925. On average there is a collector channel about every 10 to 15 degrees in the circumference of the anterior chamber (behind Schlemms canal). However the collector channels are not equal in outflow capacity. Some are larger then others, some are more or less occluded and restricted than others, there are areas where several collector channels are grouped closer together while there are other areas with very few collector channels. Furthermore many eyes, especially the ones with glaucoma have a partial or mostly collapsed Trabecular Meshwork, as shown in FIG. 27, 7945. Such a reduced or completely blocked Schlemms canal cross sections will reduce or completely stop the lateral flow (along the circumference of Schlemms canal) within Schlemms canal. If the Trabecular Meshwork is treated with any of the above described laser methods (for either tissue modulation or TM hole creation) and the laser target area falls in an area of low collector channel density or in a section where Schlemms canal is partially or fully collapsed or has a lot of anatomical tissue membranes that further reduce or stop the lateral flow, then the outflow capacity of e.g. a hole created in the Trabecular Meshwork will not lead to an efficient outflow enhancement because the outflow that now can easily go through the Trabecular Meshwork will not easily reach one or more collector channels because of these described resistances. Therefore the treatment efficiency will be diminished. If however by chance the hole is created right next or above a large collector channel (or concentrated area of collector channels), then the outflow will be less restricted and the efficacy of the intra ocular pressure reduction IOP will be greater.

The here invented method consists of:
a) determining the location of the collector channels of the to be treated eye before or while the treatment is performed.
b) Correlating the collector channel locations map from a) with one or multiple reference points in or around the eye (e.g. correlating the collector channel map with a clock hour reference where 12 o clock is on the superior point of the limbus).
c) Using the reference calibration from step b) and selecting one or multiple trabecular meshwork target areas near or right above one or multiple collector channels.
d) Aiming the treatment laser and creating the holes at the selected target areas and thereby optimizing and improving the outflow capacity of the created holes.

For point a) the determination of the collector channels is being done with one or multiple of the following methods:
Using a preoperative OCT (optical coherence Tomography) diagnostic device to find and map the location of the collector channels of the to be treated eye and if used with sufficient resolution the size of the collector channels as well. This pre-op OCT diagnostic scan can be performed long (days, weeks, . . . ) before the procedure since the collector channels are not significantly changing within weeks.
Using an OCT device that is integrated or is used in conjunction with the laser treatment system, such that it scans the eye for collector channels just prior (seconds to minutes) to the laser treatment being performed. This method is used to either find and select collector channel, near which a laser treatment is then performed or to identify and calibrate or recalibrate the location of a previously mapped collector channel (e.g. from the pre-op mapping above).
For both of the above described OCT diagnostic procedures, the OCT system can be scanned through either a goniolens (with or without integration to the laser and laser delivery system) and then through the anterior chamber towards the diagnostic target area or via a OCT system that penetrates the outer tissue layers of the eye around the limbus (with or without integration to the laser system).
Creating a momentary and sudden drop in IOP of the to be treated eye while watching the trabecular meshwork through a gonio lens view. A sudden drop in IOP will result in a blood reflex, were some blood will travel from the collector channels backwards through Schlemms canal and through the Trabecular Meshwork above and then enters the anterior chamber where it mixes with the Aqueous Humor. This blood reflex can be visibly seen and the first appearance locations on the Trabecular Meshwork are right above or near the entrances of the collector channels. These positions can then be mapped. There are several known methods available to create a sudden drop in IOP.
Using a Ultrasound biomicroscopy (UBM) system to identify and locate the collector channels.
Using a dye injected into the anterior chamber to visualize the outflow into the collector channels and by doing so mapping the location and size (outflow strength) of the collector channels.
Other imaging systems that can visualize the collector channels in an eye.

An optional tissue coagulation procedure can be applied to reduce or eliminate bleeding. See 2.e).

### 2) Opening the Entrance Area of a Collector Channels in the Eye to Treat Glaucoma:

In some glaucoma eyes the collector channels themselves have occlusions and restrictions that hinder the outflow of the Aqueous humor. In particular the entrance area of the collector channel can be partially or fully occluded by either a collapsed Schlemm's canal, a deformed or collapsed collector channel wall around the entry section or by tissue membranes and other tissue growth or debris that blocks part or all of the collector channel entrance. In this case opening a hole through Schlemm's canal even if it is placed near a collector channel will not result in good outflow.

For these cases the laser pulses are used to not only open the Trabecular Meshwork above the collector channel, but to further drill/cut an open channel into the entry section of the collector channel, see FIG. 33, 7927. The following method is here disclosed:
a) Locate one or multiple collector channels to be treated (similar to 1 a) and 1 b) above) and designate these locations to be the center of the treatment areas for the laser.
b) Aim the laser to each target area and scan the laser pulses in a way that an extended channel hole is created through the first target tissue area, here the Trabecular meshwork FIG. 33, 7907 and into the beginning section of the second target tissue area, here the collector channel. FIG. 33, 7927. (Schlemm's canal itself is not considered an target tissue layer throughout this disclosure since it is merely an open space (or sometimes collapsed and closed) through which Aqueous humor travels.
c) The width and the length of this channel can be optionally be changed and set on the laser system. The cross section of this channel can be round, oval or any other shape and can extend sideways along the Schlemms canal to the left and the right of the collector channel opening to also allow better outflow into Schlemms canal and other collector channels.
d) The laser scanning pattern may include sections where the laser spot is scanned from deeper inside the tissue (posterior, further down in the direction of the laser beam) to be opened to more shallow layers (anterior, further up the laser beam) such that the debris caused by the photo disruptive shockwaves is being pushed towards the anterior chamber and therefore minimizing occlusions inside Schlemms Canal and inside the collector channel that could be caused by this debris.
e) Optionally, a second laser source with a longer pulsed or cw laser that is not photo disruptive, but rather heats up the tissue can be used on the same target area to coagulate the tissue before it has been removed or around the remaining tissue after the channel has been opened, to minimize or stop any bleeding.

### 3) Creating Openings in the Back Wall of Schlemm's Canal in an Eye to Treat Glaucoma:

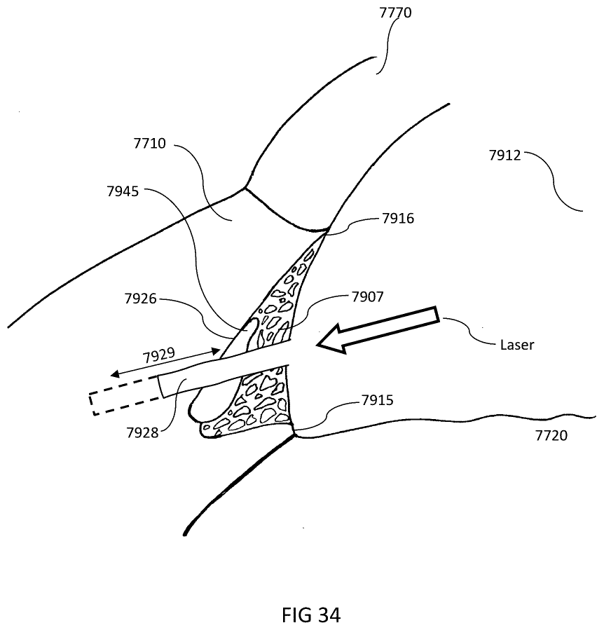

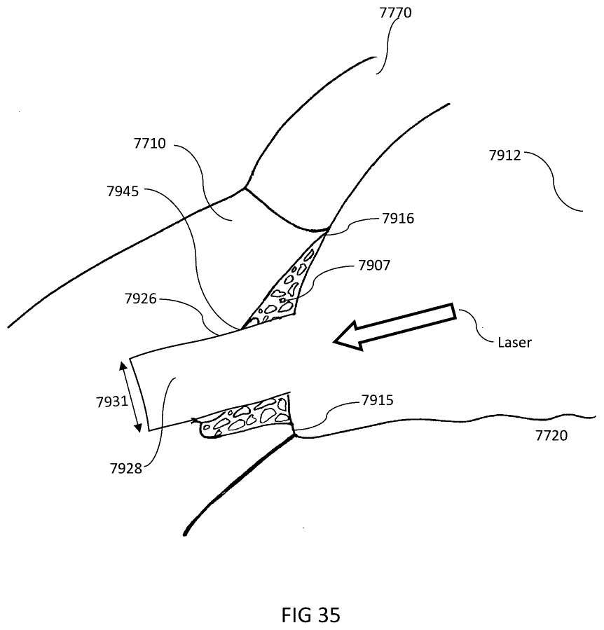

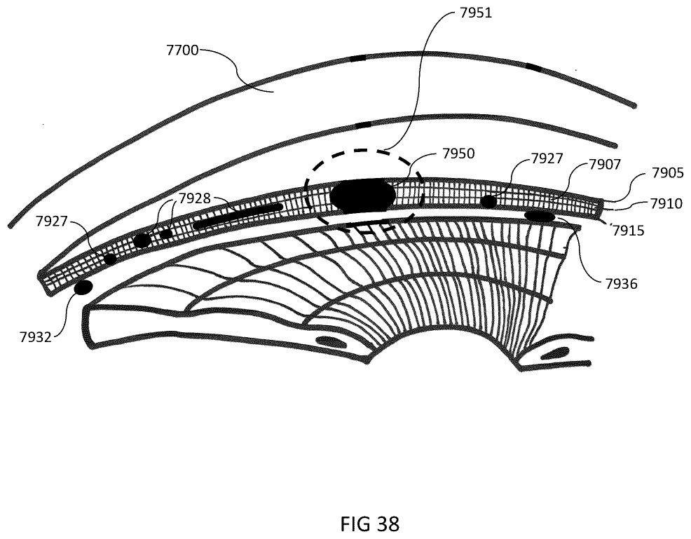

In this here disclosed method the laser pulses are used to create one or multiple channel openings FIG. 34, 7928 through first target tissue area, here the Trabecular Meshwork 7907, through Schlemm's canal 7945 and through the back wall of Schlemm's canal, 7926 and into the second target tissue area, here the scleral tissue 7710. The length 7929 and the vertical thickness FIG. 35, 7931 of the channel can vary. The length 7929 is in a range of 10 um into the sclera to all the way through the sclera (around 2 mm from Schlemm's canal). The vertical width FIG. 35, 7931 is in a range from 10 um to 500 um (micrometers). The horizontal width can be such that the opening cross section is circular or elongated along the Trabecular Meshwork 7907 circle FIG. 38, 7928.

These openings will create various novel outflow channels that will lower the IOP of a eye and therefore treat glaucoma.

This method does not require to locate or target the collector channel openings, however there is a statistical chance to randomly create a channel near or at an collector channel, depending on how many channels are cut and how large they are.

To minimize or eliminate the chance of bleeding an optional coagulation procedure as described in 2 e) above can be added.

4) Creating Openings Behind the Scleral Spur and into the Scleral Tissue of an Eye to Treat Glaucoma:

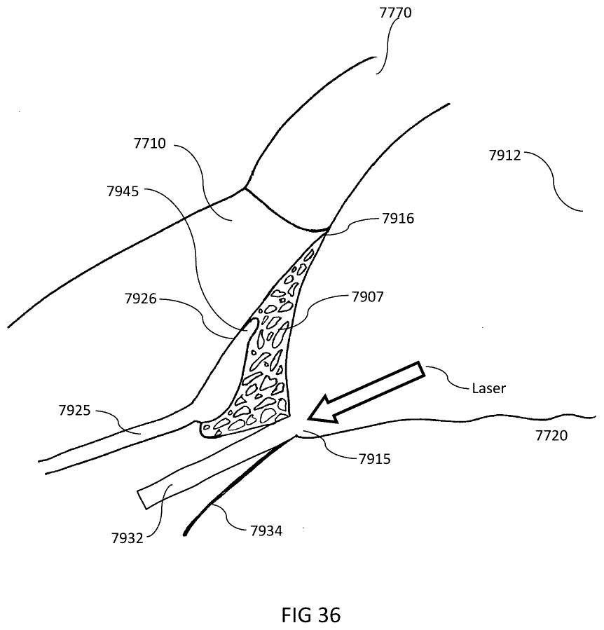

In this here disclosed method the photo disruptive laser pulses are used to create one or multiple channel openings FIG. 36, 7932 through the first target tissue area, here the region of the scleral spur 7915 (at or slightly below the bottom end of the Trabecular Meshwork 7907) and into the second target tissue area, here the scleral tissue 7710. These channels will create an increased outflow of Aqueous Humor and therefore will lower the IOP of the eye through diffusion and leakages from this new channel into nearby collector channels, into the Trabecular Meshwork from the bottom and into the choroidal tissue through the sclera to choroid transition plane 7934. These channels, from here on named Spur-Channels can vary in size, shape and penetration length as shown in FIGS. 38, 7932 and 7936.

Staying mostly above the transition line to the choroid 7934 will minimize or eliminate the chance of bleeding. Optionally a coagulation procedure as described in 2 e) above can be added.

5) Creating Openings Behind the Scleral Spur and into the Scleral Tissue of an Eye to Treat Glaucoma:

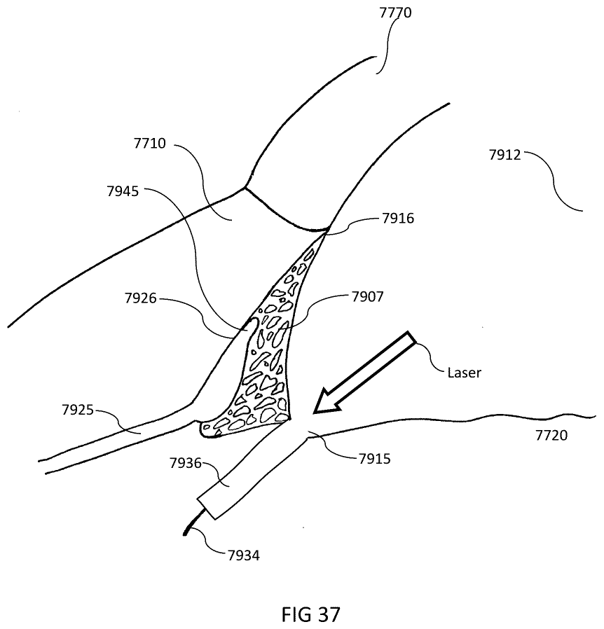

In this here disclosed method the laser pulses are used to create one or multiple channel openings FIG. 37, 7936 through the first target tissue area, here the region of the scleral spur 7915 (at or slightly below the bottom end of the Trabecular Meshwork) and into the second target tissue area, here a channel angled downwards such that the channel lays in the transition region between the sclera and choroid 7934. From here on out this channel is named Chor-Channel. The mechanism of increased outflow of Aqueous humor for the Chor-Channel is similar to the diffusion and leakage flow of the Spur-Channel, but with an increased leakage and flow part into the choroidal space. This channel is now also close and can overlap with the tissue area (ciliary body) that produces the Aqueous humor. The Chor-Channel therefore disrupts part of the Aqueous Humor production site and therefore lowers the IOP additionally by reducing Aqueous Humor production. The strength of this production lowering effect can be increased by tilting the angle of the Chor-Channel 7936 further downwards into the Choroid, by increasing its length and by creating several and or wider Chor-Channels to disrupt more of the Aqueous producing tissue area.

Cutting/drilling into Choroid tissue increases the risk of bleeding and therefore optionally a coagulation laser procedure as described in 2 e) above can be added.

FIG. 38 illustrates the various laser eye surgery methods described above. 7950 shows a large laser drilled hole through the Trabecular Meshwork 7907, e.g. for Method 1.
7927 shows various channels from Method 2.
7928 shows various channels from Method 3.
7932 shows a channel from Method 4.
7936 shows a channel from Method 5.

### Laser System Devices:

The here disclosed Methods 1 to 5 can be performed with a femtosecond photodisruptive laser system similar to the once known in femto LASIK and femto-Cataract procedure systems. The femto-laser pulses have a wavelength between 800 nm and 1500 nm, a pulse duration of 200 fs to 2000 fs, a pulse energy of 2 uJ to 100 uJ, focused to a spot size of 2-10 um spot diameter FWHM and a laser repetition firing rate of 100 Hz to 50 kH.

However, since all here described holes, channels and treated tissue areas are mostly in the 10 μm to 500 um diameter opening range, no sub 10 um precision in the cutting of the here disclosed openings is required and since furthermore a relatively large volume >10000 um\hat{ }3 (cubic micrometer) of tissue is being removed in the here disclosed methods, a longer pulsed laser with a larger spot size and a higher pulse energy is better suited to be used for these channels as long as the laser pulse peak fluence is still sufficient to achieve photo disruptive optical breakdown in the tissue area where the laser pulses are focused. The preferred laser system for the here disclosed methods 1 to 5 is therefore a nanosecond ns pulsed laser system with a pulse duration of 0.5 ns to 50 ns, a pulse energy of 50 uJ to 5 mJ, a spot size of 10 μm to 100 um and a repetition rate of single shot to 1000 Hz.

The laser parameters are optimized such that a photo disruption is still happening in the focus area of the nanosecond laser pulses (similar to the photo disruption of the femtosecond laser pulses, but with higher energy and bigger shockwaves and larger cavitation bubbles).

Not only will such a longer pulsed laser (ns-Laser) cut the tissue channels much faster compared to a femtosecond laser, but it will also reduce the cost, complexity and size of the laser system and delivery system dramatically and will therefore allow these glaucoma procedures to be available to a much larger worldwide patient population due to its significantly lower cost.

A laser system producing ns laser pulses with the specification above that is configured for photo disruptive mode in the laser focal region of the target region of an eye is hereby disclosed for use of cutting channels in an eye according to the methods 1 to 5 above.

In another embodiment, the ns laser system above is being configured in a lower pulse energy and/or larger spot size mode (compared to the photo disruptive mode above) such that the laser-tissue effect falls below the photo disruptive energy density threshold and turns into a thermal mode where the laser pulses are being absorbed by the target tissue and increase the temperature of that target tissue. These thermal mode laser pulses are used to perform the tissue coagulation as described in 2 e) and used in Method 1 to 5.

In another embodiment the here disclosed ns-laser system is being switched from Thermal mode to Photo disruptive Mode and optionally back again under the input control of an operator or with an automatic preprogrammed switching sequence, This will allow the target tissue to be coagulated before or after (or both) the channel cutting laser pulses are applied to reduce or eliminate bleeding in the eye.

1. A method for increasing aqueous humor outflow of an eye, having a cornea, an anterior chamber filled with aqueous humor, having natural outflow pathways, an anterior angle area which is including a Trabecular Meshwork, a scleral spur, a Schlemm's canal, a collector channel, scleral tissue and a transition region between a sclera and a choroid within and around the anterior angle area of the eye, the method comprising:
delivering focused photo disruptive laser pulses through the cornea into the anterior chamber and towards a first target tissue layer within the anterior angle area such that a first opening through said first target tissue layer is created and
further the focused photo disruptive laser pulses being scanned through said first opening onto a second target tissue layer within the anterior angle area such that a second opening or channel into the second target tissue layer is created and
thereby creating an additional outflow pathway for the Aqueous humor of the anterior chamber and thereby reducing the intraocular pressure of the eye.

2. A method of claim 1 where the first target tissue layer is the Trabecular Meshwork and the second target tissue layer is the collector channel.

3. A method of claim 1 where the first target tissue layer is the Trabecular Meshwork and the second target tissue layer is the scleral tissue.

4. A method of claim 1 where the first target tissue layer is the scleral spur and the second target tissue layer is the scleral tissue.

5. A method of claim 1 where the first target tissue layer is the scleral spur and the second target tissue layer is the scleral tissue.

6. A method of claim 1 where the first target tissue layer is the scleral spur and the second target tissue layer is the transition region between a sclera and a choroid.
